(19) United States
(12) Patent Application Publication
(10) Pub. No.: US 2014/0171984 A1
(43) Pub. Date: Jun. 19, 2014

(54) CLIP FOR REPAIR OF CARDIAC VALVE

(71) Applicant: Brett Allen Hillukka, Hanover, MN (US)

(72) Inventor: Brett Allen Hillukka, Hanover, MN (US)

(73) Assignee: ST. JUDE MEDICAL, INC, St. Paul, MN (US)

(21) Appl. No.: 14/235,544

(22) PCT Filed: Jul. 19, 2012
(86) PCT No.: PCT/US2012/047288

§ 371 Date: Jan. 28, 2014

Related U.S. Application Data
(60) Provisional application No. 61/512,997, filed on Jul. 29, 2011.

(51) Int. Cl. A61B 17/122

(52) U.S. Cl. CPC A61B 17/1227
USPC 606/151

(57) ABSTRACT

A clip (100, 200, 900, 1000, 1100, 1200) for repairing a cardiac valve includes an elongated body (102, 202, 902, 1132, 1202, 1302) and a locking mechanism (110, 1110, 1210, 1310). The elongated body has first and second ends and is adapted to change between an open condition and a closed condition. The locking mechanism is adapted to lock the elongated body in the closed condition. The locking mechanism may include interengaging teeth (116, 118; 212, 214) on the first end (1104, 1204, 1304) and second end (1106, 1206, 1306) of the body. Alternatively, the locking mechanism may include a return (1121, 1212, 1312) on the first end of the body sized to overlap with the second end of the body in the closed condition.

### CROSS-REFERENCE TO RELATED APPLICATIONS

This application claims the benefit of U.S. Provisional Patent Application No. 61/512,997, filed Jul. 29, 2011, the disclosure of which is hereby incorporated by reference herein.

### BACKGROUND OF THE INVENTION

The present invention is related to heart valve repair, and more particularly to devices, systems, and methods for transcatheter repair of a heart valve leaflet.

Properly functioning heart valves can maintain unidirectional blood flow in the circulatory system by opening and closing, depending on the difference in pressure on each side of the valve. The two atrioventricular valves (mitral and tricuspid valves) are multi-cusp valves that prevent backflow from the ventricles into the atria during systole. They are anchored to the wall of the ventricle by chordae tendinae, which prevent the valve from inverting.

The mitral valve is located at the gate of the left ventricle and is made up of two leaflets and a diaphanous incomplete ring around the valve, known as the mitral valve annulus. When the valve opens, blood flows into the left ventricle. After the left ventricle fills with blood and contracts, the two leaflets of the mitral valve are pushed upwards and close, preventing blood from flowing back into the left atrium and the lungs.

Mitral valve prolapse is a type of myxomatous valve disease in which the abnormal mitral valve leaflets prolapse (i.e., a portion of the affected leaflet may be billowed, loose, and floppy). Furthermore, the chordae tendinae may stretch and thus become too long, or the chordae tendinae may be ruptured. As a result, the valve does not close normally. As a result of being stretched, the unsupported valve leaflet bulges back, or “prolapses,” into the left atrium like a parachute. Thus, as the ventricle contracts, the abnormal leaflet may be propelled backwards, beyond its normal closure line into the left atrium, thereby allowing blood to flow back into left atrium and toward the lungs.

Mitral valve prolapse causes mitral regurgitation. Isolated posterior leaflet prolapse of the human heart mitral valve (i.e., prolapse of a single leaflet) is the most common cause of mitral regurgitation. The exact cause of the prolapse is not clear. Untreated mitral regurgitation may lead to congestive heart failure and pulmonary hypertension.

Despite the various improvements that have been made to devices and methods for mitral valve leaflet repair, there remain some shortcomings. For example, conventional methods of treating mitral valve prolapse include replacement of the mitral valve, clipping the two mitral valve leaflets to one another, and resection of the prolapsed segment using open heart surgery. Such surgical methods may be invasive to the patient and may require an extended recovery period.

There therefore is a need for further improvements to the current techniques for treating heart valve leaflet prolapse. Among other advantages, the present invention may address one or more of these needs.

### BRIEF SUMMARY OF THE INVENTION

The present disclosure relates to clips and fasteners for cardiac valve repair. In one embodiment, a clip for repairing a cardiac valve includes an elongated body and a locking mechanism. The elongated body has an outer surface, an inner surface, and first and second ends, and has an open condition in which the first end is spaced apart from the second end, and a closed condition in which the first end at least partially overlaps with the second end. The inner surface of the body may be roughened. The locking mechanism is adapted to lock the elongated body in the closed condition.

The locking mechanism may include first and second racks. The first rack has a plurality of first teeth protruding from the outer surface of the elongated body. The second rack has a plurality of second teeth protruding from the inner surface of the elongated body, the second teeth being configured to engage the first teeth when the elongated body is in the closed condition to lock the elongated body in the closed condition. The first teeth may protrude at an oblique angle from the outer surface of the elongated body. The second teeth may protrude at an oblique angle from the inner surface of the elongated body.

The clip may further include a plurality of protrusions on the inner surface of the elongated body. Each of the protrusions may have a substantially triangular shape. At least one of the protrusions may be shaped as an isosceles triangle. At least one of the protrusions may be shaped as a right triangle. At least one of the protrusions may have first and second sides, the first side being longer than the second side. At least one of the protrusions may have a substantially curved shape.

The clip may further include a plurality of recesses on the inner surface of the elongated body. The recesses may have a substantially round shape.

The locking mechanism may include a widened member at the first of the body, the widened member having an aperture extending therethrough. The aperture may be dimensioned to receive at least a portion of the second end of the body in the closed condition.

The body of the clip may have a generally U-shaped configuration in the closed condition, with a first leg of the body positioned alongside a second leg of the body and the first end adjacent the second end. The locking mechanism of this clip may include a return on the second end of the body, the return extending toward a closed end of the U-shaped configuration by an amount sufficient to overlap with the first end of the body to thereby hold the body in the closed configuration. The return may include a first hook and the first end of the body may include a second hook, the first hook being adapted to engage the second hook to hold the body in the closed condition. The first end of the body may include a shaped member, and the return may include a recess sized and shaped to receive the shaped member in assembled relationship.

### BRIEF DESCRIPTION OF THE DRAWINGS

Various embodiments of the present invention will now be discussed with reference to the appended drawings. It is appreciated that these drawings depict only some embodiments of the invention and are therefore not to be considered limiting of its scope.

FIG. 1 is a diagrammatic view of a mitral valve, with the anterior leaflet partially broken away;

FIG. 2A is a perspective view of the distal portion of one embodiment of a device for transcatheter gathering of heart valve leaflet tissue, engaged with the posterior leaflet of a mitral valve;

FIG. 2B a is perspective view of the distal portion of the device of FIG. 2A with portions removed to illustrate the interior thereof;

FIG. 2C is a view similar to FIG. 2B, but shown with the retaining arm in a partially-retracted position;

FIG. 2D is a view similar to FIG. 2B, but shown with the retaining arm in the retracted position;

FIG. 2E is a view similar to FIG. 2B, but shown with the clip in a deployed position;

FIG. 3A is a front perspective view of a clip for repairing a cardiac valve;

FIG. 3B is a top view of the clip of FIG. 3A in an open condition;

FIG. 3C is a top view of the clip of FIG. 3A in a closed condition;

FIG. 4 is a front perspective view of another embodiment of a clip for repairing a cardiac valve;

FIG. 5 is a rear perspective view of the clip of FIG. 4;

FIGS. 6-11 are partial longitudinal cross-sectional views showing a plurality of different surface configurations for the clips of the present invention;

FIG. 12 is a front perspective view of a further embodiment of a clip for repairing a cardiac valve;

FIG. 13 is a front perspective view of a variant of the clip of FIG. 12;

FIG. 14 is a top view of yet another embodiment of a clip for repairing a cardiac valve in an open condition;

FIG. 15 is a top view of the clip of FIG. 14 in a closed condition;

FIG. 16 is a side perspective view of a still further embodiment of a clip for repairing a cardiac valve in an open condition;

FIG. 17 is a side perspective view of the clip of FIG. 16 in a closed condition; and

FIG. 18 is an enlarged partial top view of an embodiment of a locking mechanism for a clip designed for repairing a cardiac valve.

### DETAILED DESCRIPTION

The present invention relates to clips or fasteners for repairing a cardiac valve. In the present disclosure, the clip is described in connection with the repair of a mitral valve leaflet, but it may be useful in the repair of other types of cardiac valves or other types of loose body tissue. The embodiments of the clip described herein may be used with any suitable instrument or device capable of applying the clip.

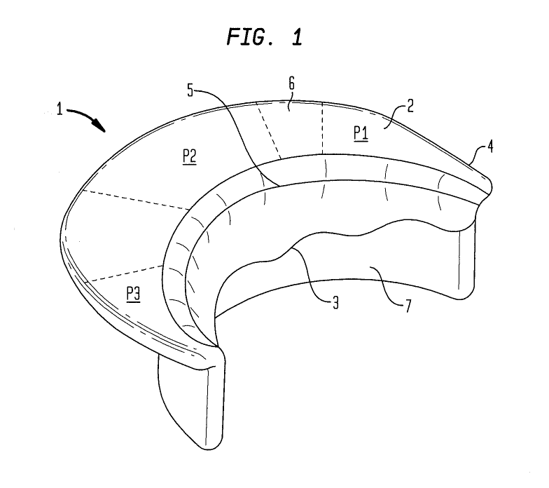

Referring to FIG. 1, an exemplary mitral valve 1 includes a posterior leaflet 2 and an anterior leaflet 3. The leaflets 2 and 3 extend from an annulus 4 to a coaption line 5 where the leaflets meet. The posterior leaflet 2 has an upper portion 6 that is generally perpendicular to the direction of blood flow through the valve 1 and that extends between the annulus 4 and the coaption line 5. Additionally, the posterior leaflet 2 has a lower portion 7 that is generally parallel to the direction of blood flow through the valve 1 and that extends below the coaption line 5. The posterior leaflet 2 has three scalloped portions P1, P2, and P3, any of which may include a portion that is billowed, loose, or floppy, and therefore be the cause of a prolapse condition of the valve. The inventive devices, systems, and methods described herein may be adapted to repair such a billowed, loose, or floppy portion of the posterior leaflet 2 or the anterior leaflet 3 by applying one or more clips to the same.

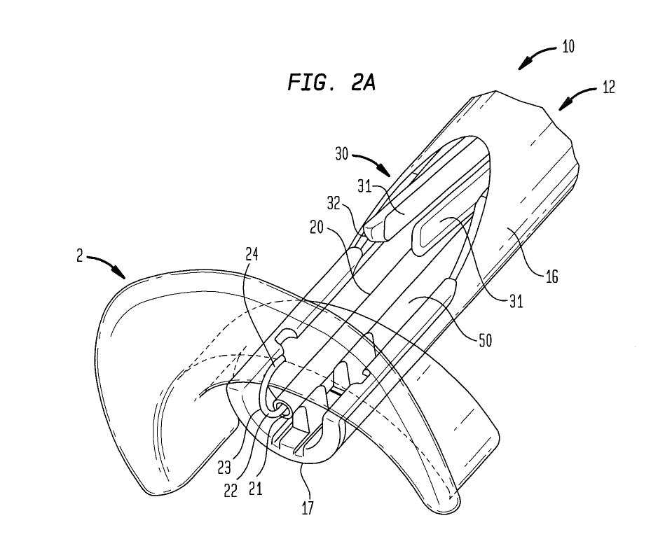

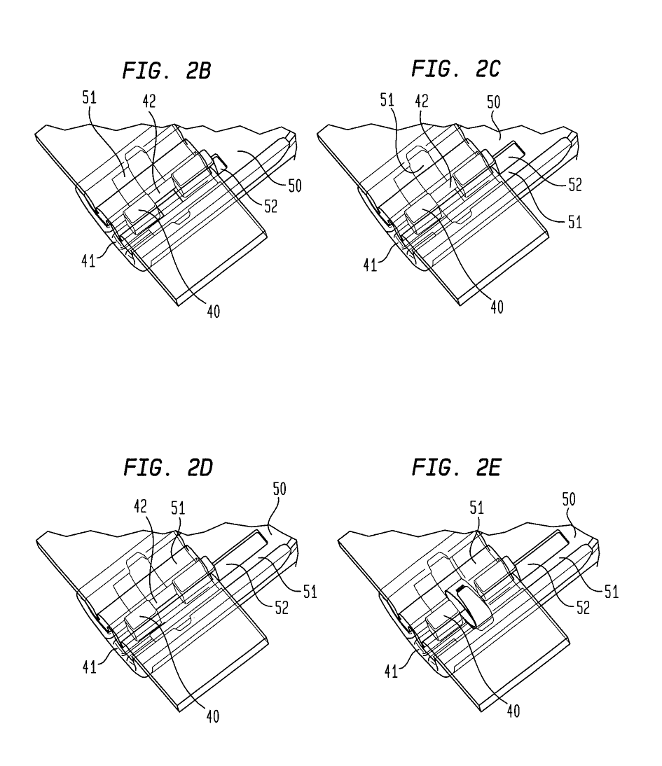

An exemplary device for applying such clips is described in U.S. Provisional Patent Application No. 61/438,446 titled “Apparatus and Method for Heart Valve Repair” and filed on Feb. 1, 2011, the entire disclosure of which is incorporated herein by reference. FIG. 2A shows a distal portion of this exemplary device 10 grasping a mitral valve leaflet 2. The device 10 includes a handle (not shown) for controlling the device and an elongated operating assembly 12 adapted to be inserted through the apex of a human heart so that a distal portion of the assembly can reach the patient's mitral valve leaflet 2 for repair thereof.

The operating assembly 12 is operative to capture the mitral valve leaflet 2 and create a fold or pleat in the same prior to the application of a clip to the folded tissue. The operating assembly 12 includes a containment tube 20 disposed within an outer tube 16 and longitudinally slidable therein between a retracted position within the outer tube and a deployed position in which a distal tip 21 of the containment tube protrudes distally beyond the distal edge 17 of the outer tube. A capture tool in the form of a grasping wire 22 is longitudinally slidable within the containment tube 20 between a retracted position substantially entirely within the lumen of the containment tube, and a deployed position in which a distal portion 23 of the grasping wire protrudes from the distal tip 21 of the containment tube. The grasping wire 22 may have a substantially linear configuration when fully retracted within the containment tube 20 and the distal portion 23 thereof may assume the shape of a hook 24 when deployed from the containment tube. As will be explained below, the hook 24 is adapted to grasp the tissue of the mitral valve leaflet 2 and draw it towards the distal end of outer tube 16. A fork 30 having a pair of spaced apart tines 31 may be longitudinally slidable within the outer tube 16 between an initial or retracted position and positions at which the fork captures and clamps the leaflet tissue so as to form a fold or pleat therein.

As seen in FIGS. 2A-2E, the device 10 further includes a tissue support in the form of an anvil 40 mounted on the closed side 41 of the outer tube 16 so as to lie between the closed side 41 and the containment tube 20 when the containment tube is in the deployed position. The anvil 40 has a proximal portion and a distal portion, with a gap 42 defined therebetween for receiving and holding a clip to be applied to the leaflet tissue. The widths of the proximal and distal portions are such that the anvil 40 may be received between the tines 31 of the fork 30 during the use of the device 10 to repair the valve leaflet.

A retaining arm 50 of the operating assembly 12 is disposed within the outer tube 16 and is longitudinally slidable therein between an initial position (FIG. 2B) and a retracted position (FIG. 2D). The retaining arm 50 includes a pair of fingers 51 separated by an elongated slot 52 to receive the anvil 40 when the retaining arm 50 is in the initial position. In this initial position, the fingers 51 lie on either side of the anvil 40 and engage the clip disposed within the gap 42, holding it in place against the closed side of 41 of the outer tube 16. The retraction of the retaining arm 50 releases the clip for application to tissue.

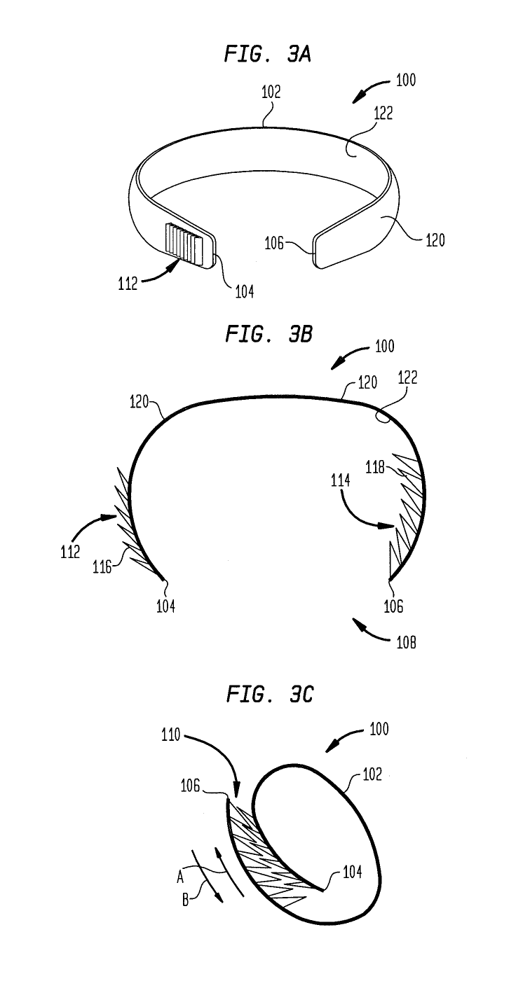

FIGS. 3A-3C illustrate one embodiment of a clip or fastener 100 for repairing a cardiac valve. The clip 100 has an elongated body 102 which terminates in ends 104 and 106. The body 102 is adapted to move from an open condition shown in FIGS. 3A and 3B, in which the ends 104 and 106 are spaced apart to enable the clip to receive the leaflet tissue, to a closed position shown in FIG. 3C, in which the ends 104 and 106 overlap to securely hold the captured leaflet tissue. To this end, the elongated body 102 may be wholly or partly made of any suitable resilient or shape memory biocompatible material, and may include materials that are bioabsorbable or biodegradable. Suitable biocompatible shape memory materials include, but are not limited to, shape memory alloys, such as nitinol and nickel-titanium-cobalt alloys, and shape memory polymers, including those based on polyetheretherketone (PEEK) and/or polymethylmethacrylate (PMMA). Suitable biocompatible resilient materials include, but are not limited to, titanium alloys, cobalt chrome alloys and stainless steel alloys.

The clip 100 is formed so as to be biased to the closed condition. That is, without any external force exerted on the clip, the ends 104 and 106 of the clip will overlap with one another so as to form a closed annular structure. The clip 100 may be deformed to the open condition when loaded into the delivery device. More particularly, the ends 104 and 106 of the clip may be moved away from one another to define a gap 108 therebetween, such that clip 100 will have an open annular structure. The retaining arm 50 of the delivery device 10 may then be slid over the clip 100 to hold it in this open condition. When it is desired to deploy clip 100, the retaining arm 50 may be withdrawn, whereupon the biasing force of the clip will return it to the closed condition.

The clip 100 may further include a locking mechanism for maintaining the clip in the closed condition. Each of the embodiments of the clip described herein are substantially similar to one another, differing primarily in the structure of the locking mechanism each employs.

One embodiment of a locking mechanism according to the present invention is the ratcheting mechanism 110 shown in FIGS. 3A-3C. The ratcheting mechanism 110 may include a first rack 112 disposed on an outer surface 120 of the elongated body 102 adjacent the first end 104 and a second rack 114 disposed on an inner surface 122 of the elongated body adjacent the second end 106. The first rack 112 may include a plurality of teeth 116 protruding at an oblique angle from the outer surface 120 of the elongated body 102, whereas the second rack may include a plurality of teeth 118 protruding at an oblique angle from the inner surface 122 of the elongated body. The teeth 116 and 118 are preferably angled in opposite directions so that they can slide against one another without interference when the clip 100 is moved in the closing direction (i.e., the direction of arrow A in FIG. 3C), but will interfere and mate with one another when attempts are made to move the clip to the open condition (i.e., the direction of arrow B in FIG. 3C). That is, the teeth 118 of the second rack 114 are dimensioned and oriented to engage the teeth 116 of the first rack 112 to lock the clip 100 in the closed condition, as shown in FIG. 3C. The teeth 116 and 118 may be substantially triangular when viewed in a direction normal to the plane in which the clip lies and may be made of the same material as the elongated body 102. Alternatively, the teeth 116 and 118 and the elongated body 102 may be formed from different materials.

The clip 100 or any other clips or fasteners described herein may be used for repairing a cardiac valve. More particularly, the clip 100 may be deployed, such as with the device 10 described above, to hold the tissue of a valve leaflet in a pleated or folded configuration. When the retaining arm 50 is retracted to deploy the clip 100, the clip automatically moves from the open condition to the closed condition with a strong biasing force. As it closes, the clip 100 encompasses tissue of the leaflet, while the ends 104 and 106 of the elongated body 102 pierce the leaflet tissue and overlap with one another. The overlapping of the ends 104 and 106 of the elongated body 102 enables the teeth 116 of the first rack 112 to mate with the teeth 118 of the second rack 114 to lock the clip 100 in the closed condition.

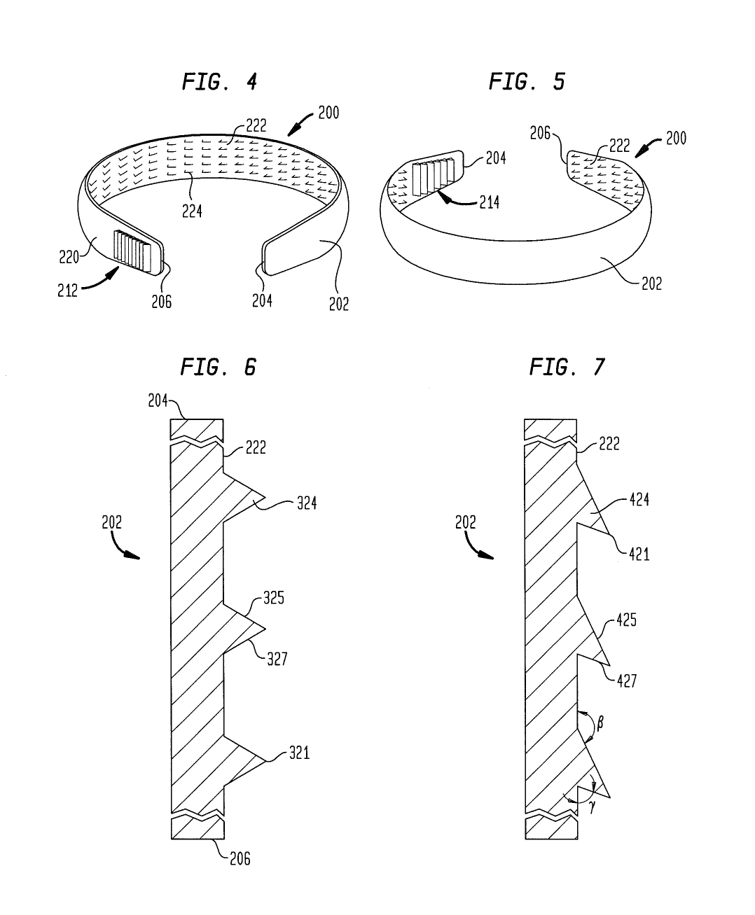

FIGS. 4 and 5 show an alternate embodiment of a clip 200 of the present invention. The clip 200 is substantially similar to the clip 100 shown in FIGS. 3A-C, and includes first and second racks of teeth 212, 214, respectively. In this embodiment, however, the clip 200 may additionally have a roughened inner surface 222 for increasing the friction between the clip 200 and the captured tissue. The roughened surface may be formed by a plurality of protrusions 224 projecting from the inner surface 222 of the clip 200. The protrusions 224 may extend in one or more longitudinal rows from the second end 206 of the elongated body 202 of the clip to a location adjacent the second rack 214. The longitudinal rows may be substantially linear or sinusoidal, and may be continuous or discontinuous. Moreover, the protrusions 224 may be disposed along only a central portion of the inner surface 222 between the first and second ends 204 and 206 of the elongated body 202, or only on the portion of the inner surface 222 opposite the location of the first rack 212.

The protrusions 224 may have different shapes and sizes, and the protrusions on any one clip 200 may be the same or different. FIG. 6 illustrates an example of triangularly-shaped protrusions 324 that may be incorporated into the clip 200 or any other clip described herein. Specifically, each of the protrusions 324 may be shaped substantially as an isosceles triangle having a first side 325 facing the first end 204 of the elongated body 202 and a second side 327 facing the second end 206 of the elongated body. The first and second sides 325 and 327 of each protrusion 324 have substantially similar lengths and are oriented at the same or complementary oblique angles relative to the inner surface 222 of the elongated body 202. For example, the first side 325 may be oriented at about 135 degrees relative to the inner surface 222 of the elongated body 202, and the second side 327 may be oriented at about 135 degrees in the opposite direction with respect to the inner surface of the elongated body. Each protrusion 324 may have a sharp pointed end 321 that may become embedded in the tissue captured by the clip.

Another example of triangularly-shaped protrusions 424 that may be incorporated into the clip 200 or any other clip described herein is depicted in FIG. 7. The protrusions 424 are similar to the protrusions 324 shown in FIG. 6, but the lengths of the first and second sides 425 and 427 of each protrusion 424 may not be equal, such that the angles formed between the first and second sides and the inner surface 222 of the elongated body 202 are not equal or complementary. Thus, the first side 425 of each protrusion 424 may be longer than the second side 427 such that the oblique angle β defined between the first side 425 and the inner surface 222 of the elongated body 202 is larger than the oblique angle γ defined between the second side 427 and the inner surface 222. Each protrusion 424 may have a sharp pointed end 421 that may become embedded in the tissue captured by the clip.

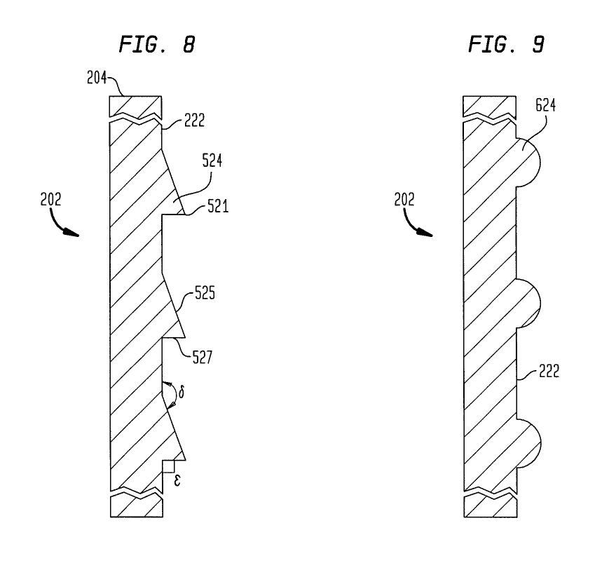

FIG. 8 shows another example of triangularly-shaped protrusions 524 that may be incorporated into the clip 200 or any other clip described herein. The protrusions 524 are similar to the protrusions 424 seen in FIG. 7, but are substantially shaped as right triangles. The first side 525 of each protrusion 524 may be longer than the second side 527 and may be oriented at an oblique angle δ relative to the inner surface 222 of the elongated body 202. The second side 527 of each protrusion 524 may be oriented at a right angle ε (i.e., 90 degrees) relative to the inner surface 222 of the elongated body 202. Each protrusion 524 may have a sharp pointed end 521 that may become embedded in the tissue captured by the clip.

FIG. 9 illustrates another example of protrusions 624 that may be incorporated into the clip 200 or any other clip described herein. The protrusions 624 may extend from the inner surface 222 of the elongated body 202 of the clip in a manner similar to the protrusions described above, but each of the protrusions 624 may have a substantially curved shape, preferably a smoothly curved shape. The lack of sharp edges on protrusions 624 may minimize trauma to the captured tissue. In one specific embodiment, each of the protrusions 624 may have a substantially semi-circular shape.

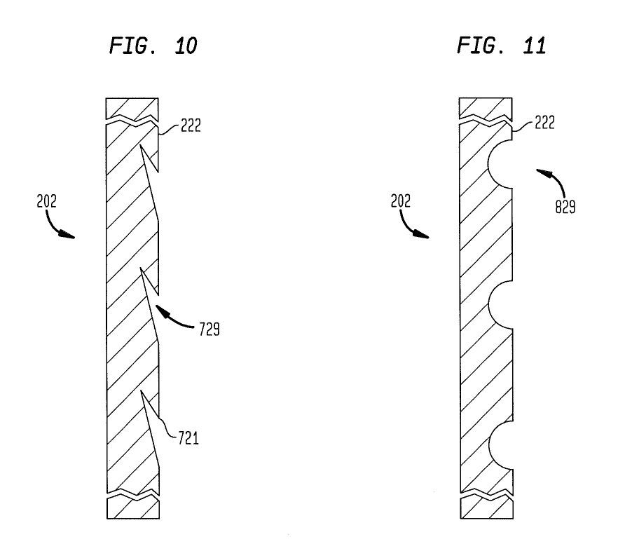

FIG. 10 depicts another example of a surface configuration that may be incorporated into the clip 200 or any other clip described herein. In this surface configuration, the inner surface 222 of the clip 200 has a plurality of recesses 729 for increasing the friction between the clip and the captured tissue. Each of the recesses 729 may be undercut with a substantially triangular shape, thereby forming a sharp edge 721 on the inner surface 222 of the elongated body 202. The sharp edges 721 may become embedded in the tissue captured by the clip.

FIG. 11 shows another embodiment of a clip having recesses 829 on the inner surface 222 of elongated body 202, rather than protrusions, for increasing the friction between the clip and the captured tissue. Although the figure shows substantially spherical or round recesses 829 on the inner surface 222 of the elongated body 202, the recesses may have other suitable shapes or configurations. The recesses 829 may be incorporated on the inner surface of any of the clips described herein, either in lieu of or in addition to the protrusions described above. The recesses 829 may extend in one or more longitudinal rows from the second end 206 of the elongated body 202 of the clip to a location adjacent the second rack 214. The longitudinal rows may be substantially linear or sinusoidal, and may be continuous or discontinuous. Moreover, the recesses 829 may be disposed along only a central portion of the inner surface 222 between the first and second ends 204 and 206 of the elongated body 202, or only on the portion of the inner surface 222 opposite the location of the first rack 212. Other arrangements are also contemplated. When the clip is applied to the captured tissue, some portions of the captured tissue enter the recesses 829, thereby increasing the friction between the inner surface 222 and the captured tissue.

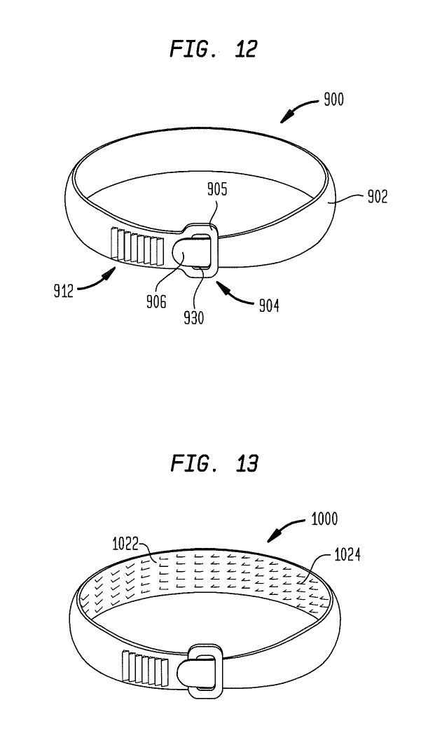

FIG. 12 illustrates a clip 900 substantially similar to the clip 100 shown in FIGS. 3A-3C, except that the elongated body 902 of the clip 900 has a widened member 905 at a first end 904 with an enlarged aperture 930 extending through the widened member. The aperture 930 is dimensioned to receive the second end 906 of the elongated body 902 and at least the portion of the elongated body supporting the second rack (not shown).

The clip 900 may be used for cardiac valve repair in a manner similar to the clip 100 described above. In this embodiment, when the clip 900 changes from the open condition to the closed condition, the second end 906 of the elongated body 902 passes through the aperture 930 of the widened member 905, thereby holding the first rack 912 in locked engagement with the second rack (not shown). During deployment, the device 10 or any other suitable device or instrument may guide the second end 906 of the elongated body 902 so that it passes through the aperture 930 of the widened member 905 as the clip 900 moves to the closed condition.

FIG. 13 shows a clip 1000 substantially similar to the clip 900. The clip 1000 may further have a roughened inner surface 1022, which may include a plurality of protrusions and/or recesses 1024. Any of the protrusions and/or recesses described above may be incorporated in the clip 1000. The clip 1000 may be used for cardiac valve repair in a manner similar to each of the clips described above.

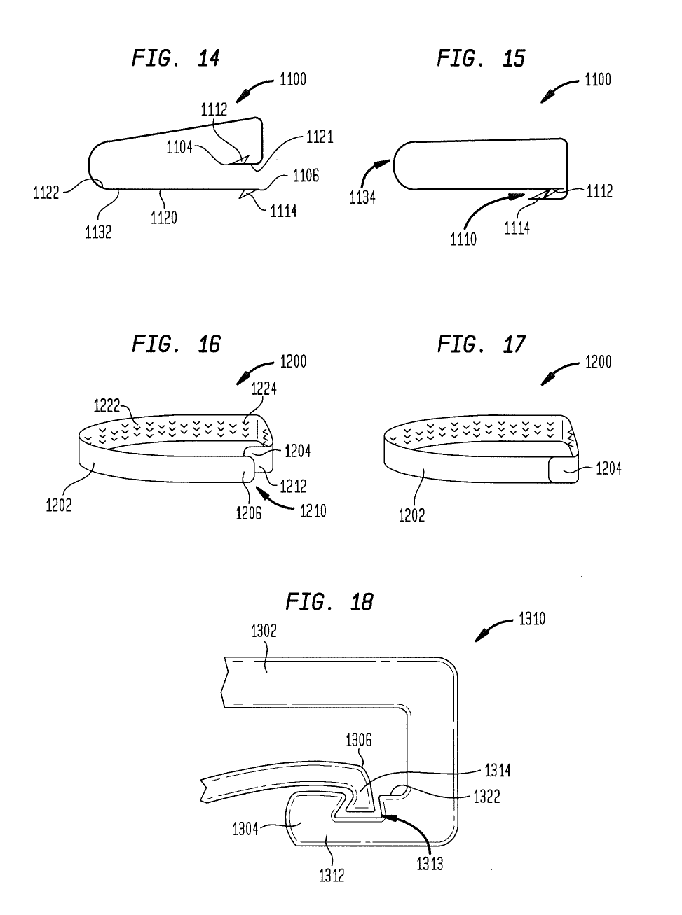

FIGS. 14 and 15 show another embodiment of a clip 1100 similar to the clip 100 described above. However, rather than a plurality of interengaging teeth, the clip 1100 may include a cantilevered snap-fit mechanism 1110 for locking the clip in its closed condition. The clip 1100 may include an elongated body 1132 having first and second ends 1104 and 1106. A first hook 1112 may be provided on an inner surface 1122 of a return 1121 at the first end 1104, and a second hook 1114 may be provided on an outer surface 1120 of the body at the second end 1106 such that the hooks face toward one another in a closed condition of the clip.

The clip 1100 may be used for cardiac valve repair in a manner similar to the clip 100 described above. When the clip 1100 is released by the deployment device for deployment onto the captured tissue, the clip will be biased from an open condition in which the first end 1104 and the second end 1106 of the body 1132 are spaced apart from one another by a substantial distance to permit the leaflet tissue to be drawn toward a middle portion 1134 of the body between the ends, to a closed condition in which the body has a generally U-shape and the ends 1104 and 1106 overlap one another, as seen in FIG. 15. If the biasing force is sufficiently strong, the ends of the body will move towards one another until the second end 1106 snaps behind the first end 1104 and hooks 1112 and 1114 engage one another, locking the clip in its closed condition. Should the biasing force be insufficient to lock the ends of the body together, the fork 30 of the delivery device 10 may be used to manually squeeze the ends 1104 and 1106 together until the first hook 1112 lockingly engages the second hook 1114.

FIGS. 16 and 17 show another embodiment of a clip 1200 substantially similar to the clip 1100 described above. In this embodiment, the clip 1200 may further include any of the protrusions and/or recesses described above. The protrusions and/or recesses 1224 of clip 1200 may be located on the inner surface 1222 of the elongated body 1202 of the clip. The snap-fit mechanism 1210 of the clip 1200 may not necessarily include hooks. Rather, in this snap-fit mechanism 1210, the first end 1204 of the elongated body 1202 has a return 1212, and the second end 1206 of the elongated body is adapted to snap behind the return to lock the clip 1200 in the closed position, as shown in FIG. 17. Accordingly, when clip 1200 is deployed onto captured tissue, the clip will move from the open condition in which the first end 1204 of the elongated body 1202 is spaced from the second end 1206 of the elongated body, toward the closed condition in which the ends 1204 and 1206 are adjacent to one another, as seen in FIG. 16. If the clip 1200 is subject to a sufficient biasing force, the second end 1206 of the elongated body will snap behind return 1212, as shown in FIG. 17, to lock the clip in the closed condition. When the biasing force is insufficient, the fork 30 of the delivery device 10 may be used to apply the additional force necessary to snap the second end 1206 of the elongated body behind the return 1212 to lock the clip 1200 in the closed condition.

FIG. 18 shows an alternate embodiment of a snap-fit mechanism 1310 that may be incorporated into the clips 1100 and 1200 or any of the other clips described herein. The snap-fit mechanism 1310 is similar to the snap-fit mechanism 1110 but does not include a pair of hooks at the ends of the elongated body of the clip. Rather, the snap-fit mechanism 1310 may include a shaped member 1314 at the second end 1306 of the elongated body 1302 and facing away from the first end 1304 prior to locking. The snap-fit mechanism 1310 may further include a return 1312 on the first end 1304 of the elongated body 1302, with a recess 1313 formed in a surface of the return facing away from the second end 1306 prior to locking. The shaped member 1314 may taper outwardly toward its free end, while the recess 1313 may be undercut with a similar shape, such that the recess will deform slightly as the shaped member is inserted therein.

The snap-fit mechanism 1310 may be used in a manner similar to the snap-fit mechanism 1110 described above. In this embodiment, however, as the clip changes from the open condition to the closed condition, the shaped member 1314 may snap into the recess 1313 to lock the clip in its closed condition, as shown in FIG. 18. As with snap-fit mechanism 1110, the fork 30 of the delivery device 10 may be used to apply any additional force that may be necessary to snap the shaped member 1314 into the recess 1313.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

### INDUSTRIAL APPLICABILITY

The clips of the present invention enable gathered biological tissue, including heart valve leaflet tissue, to be securely held in a gathered condition.

1. A clip for repairing a cardiac valve, comprising:
an elongated body having an outer surface, an inner surface, and first and second ends, the elongated body having an open condition in which the first end is spaced apart from the second end, and a closed condition in which the first end at least partially overlaps with the second end; and
a locking mechanism adapted to lock the elongated body in the closed condition.

2. The clip according to claim 1, wherein the inner surface of the body is roughened.

3. The clip according to claim 1, further comprising a plurality of protrusions on the inner surface of the elongated body.

4. The clip according to claim 3, wherein each of the protrusions has a substantially triangular shape.

5. The clip according to claim 4, wherein at least one of the protrusions is shaped as an isosceles triangle.

6. The clip according to claim 4, wherein at least one of the protrusions is shaped as a right triangle.

7. The clip according claim 4, wherein at least one of the protrusions has first and second sides, the first side being longer than the second side.

8. The clip according to claim 3, wherein at least one of the protrusions has a substantially curved shape.

9. The clip according to claim 1, further comprising a plurality of recesses on the inner surface of the elongated body.

10. The clip according to claim 9, wherein the recesses have a substantially round shape.

11. The clip according to claim 1, wherein the locking mechanism includes:
a first rack having a plurality of first teeth, the first teeth protruding from the outer surface of the elongated body; and
a second rack having a plurality of second teeth, the second teeth protruding from the inner surface of the elongated body, the second teeth being configured to engage the first teeth when the elongated body is in the closed condition to lock the elongated body in the closed condition.

12. The clip according to claim 11, wherein the first teeth protrude at an oblique angle from the outer surface of the elongated body.

13. The clip according to claim 11, wherein the second teeth protrude at an oblique angle from the inner surface of the elongated body.

14. The clip according to claim 11, wherein the locking mechanism further includes a widened member at the first end of the body, the widened member having an aperture extending therethrough, the aperture being dimensioned to receive at least a portion of the second end of the body in the closed condition.

15. The clip according to claim 1, wherein the body has a generally U-shaped configuration in the closed condition, with a first leg of the body positioned alongside a second leg of the body and the first end adjacent the second end, the locking mechanism including:
a return on the second end of the body, the return extending toward a closed end of the U-shaped configuration by an amount sufficient to overlap with the first end of the body to thereby hold the body in the closed condition.

16. The clip according to claim 15, wherein the return includes a first hook and the first end of the body includes a second hook, the first hook being adapted to engage the second hook to hold the body in the closed condition.

17. The clip according to claim 15, wherein the first end of the body includes a shaped member, and the return includes a recess sized and shaped to receive the shaped member in assembled relationship.
